(19) United States
(12) Patent Application Publication
(10) Pub. No.: US 2015/0018732 A1
(43) Pub. Date: Jan. 15, 2015

(54) Hemiparesis Subluxation Sling

(71) Applicant: Robert Thornton, Bunker Hill, WV (US)

(72) Inventor: Robert Thornton, Bunker Hill, WV (US)

(21) Appl. No.: 13/941,622

(22) Filed: Jul. 15, 2013

(51) Int. Cl. A61F 5/37

(52) U.S. Cl. CPC A61F 5/3746
USPC 602/4

(57) ABSTRACT

This sling is designed to help reduce shoulder subluxation and forward shoulder roll in patients with shoulder problems By using the uninjured arm to support the weight of the injured arm through two arm cuffs and semi-elastic straps that cross over the back this sling was specially designed to be worn under clothing like a coat, to be slept in, to be worn while preforming daily task/therapy while holding the shoulder joint comfortably in place and still allowing full range of motion in both arms and hands

### BACKGROUND

This invention relates to an arm sling, and more particularly to a Hemiparesis Subluxation Sling having adjustable means to support a humerus by lifting and holding it comfortably, without limiting the patient's range of motion.

### PRIOR ART

Heretofore various devices for stabilizing a joint, such as the shoulder joint, have been proposed.

One example is disclosed in U.S. Pat. No. 4,735,198 which defines therein a body harness comprising a torso fitting portion and an appendage fitting portion secured at one end to the torso fitting portion and having the other end thereof as a free end allowing for limited mobility of the appendage to which it is fitted. At least one elastic strap extends from and is releaseably secured to and between the torso fitting portion and the appendage fitting portion and controls mobility of the appendage.

Other shoulder motion restricting structures are disclosed in U.S. Pat Nos. 3,324,851; 3,970,316; 4,610,244; and 4,905,713.

These prior art restraining devices are all cumbersome, uncomfortable and cause a restriction which does not allow full potential rehabilitation for stroke and TBI patients

As will be described in greater detail hereinafter, the harness of the present invention is light-weight, offers a greater range of motion, and provides means for exerting a force upon an arm of the wearer which maintains the shoulder joint intact

The recovery of a patient following a stroke or TBI is affected by the extent of the brain damage and the development of complications. Development of pain in the shoulder of a hemipalegic patient is a significant and serious problem, because it can limit the patient's ability to reach his or her maximum rehabilitation potential. Shoulder pains have been identified, such as immobilization of the upper extremity, trauma of the joint structure, including brachial plexus injuries and subluxation of the gleno-humeral joint; or it may be a combination of these symptoms.

### BRIEF SUMMARY OF INVENTION

For a sling to meet the needs of the patient it has to conform to criteria considerations such as Subluxation reduction, Easily worn/put on, Ability to wear under clothing comfortably, Weight distribution, Freedom of movement of both upper and lower parts of the arm while keeping the shoulder in place. Our sling meets all these requirements. Accordingly, applicant has provided an adjustable hemi-arm sling for carrying the humerus, lifting to provide the proper position of the gleno-humeral joint.

It is an object of this invention to provide a subluxation sling for positioning of the humerus in the gleno-humeral joint without restricting circulation, range of motion or creating unnecessary pressure against the neck, armpit or ribcage.

It is another object of this invention to provide a subluxation sling that starts with a comfortable arm support. An elastic arm band with attached non-elastic cloth ends, with elastic hook and loop fastening system for arm size and comfort, is placed over the bicep of each arm. Each arm support has a strap permanently mounted on the top front which runs over the shoulder and through a quick release buckle on the rear of the opposing arm cuff, which can be adjusted toward proper lift. The third strap runs across the back from arm pit to arm pit through a plastic slide on each cuff, with a hook and loop adjustment for lateral support of the shoulder joint. The straps are made of semi-elastic material capable of receiving velcro hook, making the straps adjustable to their entire length due to their hook-tab ends.

### BRIEF DESCRIPTION OF THE SEVERAL VIEWS OF THE DRAWING

Sheet 1 contains FIGS. A, B and C. Sheet 2 contains FIG. D.

FIG. A is a view of the present invented sling, shown unobtrusively from the bodily front.

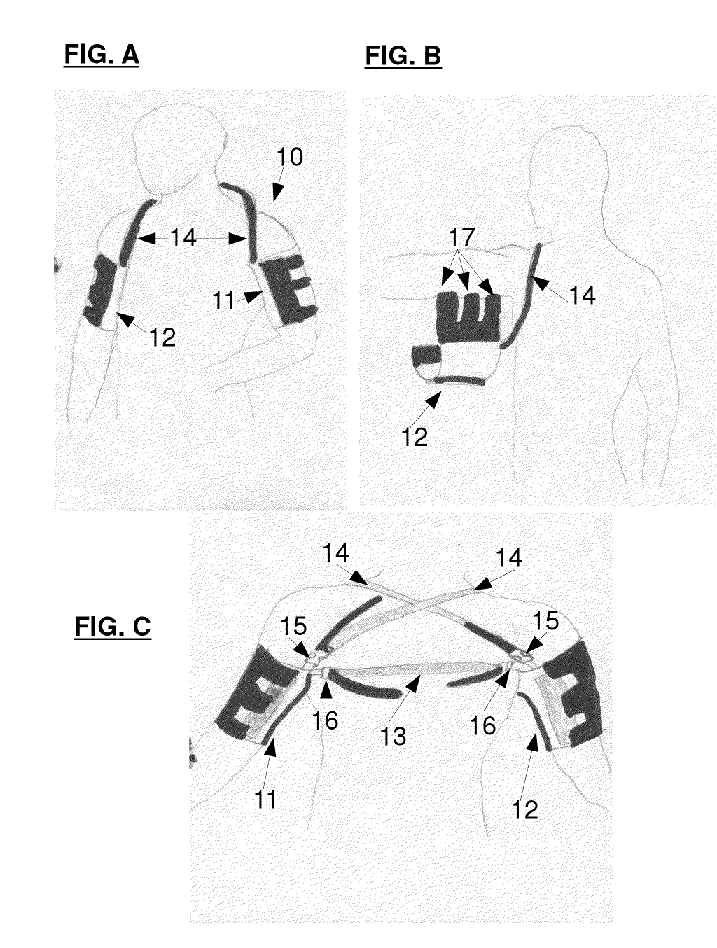

FIG. B is also a frontal view similar to FIG. 1., but shows how a arm cuff is properly applied, with shoulder strap toward the front, and the cuff tongues overlapping the arm away from the torso.

FIG. C is a view of the present invented sling, shown from the bodily rear; showing the proper path of each strap through slides and quick-release buckles, the fixture of straps back to themselves making them adjustable to their full length.

FIG. D is an exploded view of the structures forming the harness.

### DETAILED DESCRIPTION OF THE DRAWINGS

Referring now to the drawings in greater detail, there is illustrated therein the sling of the present invention generally identified in whole by reference numeral 10.

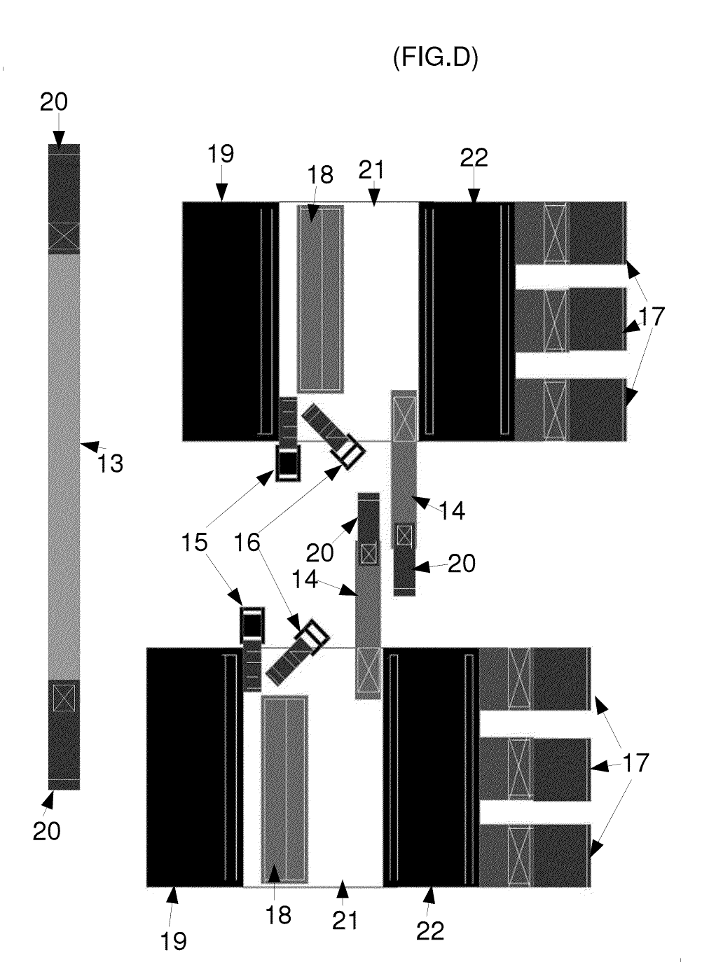

FIG. A—The sling 10 includes one left-arm cuff 11, one right-arm cuff 12 and one back-strap 13 (as shown in FIGS. 3 and 4)

FIG. B—The open left-arm cuff 11 (as shown in FIG. 4) is applied to the patient's left-arm with cuff loop 18 facing the torso. The shoulder strap 14 should be facing the front of the torso, and the quick-release buckle 15 and back-strap slide 16 should be facing the rear. The three cuff tongues 17 wrap over the arm, overlapping the opposing cloth end 19 of the left-arm cuff 11, outward away from the torso, securing all three cuff tongues 17 to the cuff loop 18.

The open right-arm cuff 12 (as shown in FIG. 4) is applied to the patient's right-arm with cuff loop 18 facing the torso. The shoulder strap 14 should be facing the front of the torso, and the quick-release buckle 15 and back-strap slide 16 should be facing the rear. The three cuff tongues 17 wrap over the arm, overlapping the opposing cloth end 19 of the right-arm cuff 12, outward away from the torso, securing all three cuff tongues 17 to the cuff loop 18.

FIG. C—The shoulder strap 14 of the left-arm cuff 11 should continue up over the left-shoulder, run through the quick-release buckle 15 on the rear of the right-arm cuff 12, then be fixated back unto itself. The shoulder strap 14 has a velcro-hook tab 20 attached with a framed-X pattern stitch, overlapping the loop side of the shoulder strap 14 with the hook of the tab 20 facing the same direction of the shoulder strap 14 loop. This makes the strap adjustable by attaching back to itself across its entire length.

The shoulder strap 14 of the right-arm cuff 12 should continue up over the right-shoulder, run through the quick-release buckle 15 on the rear of the left-arm cuff 11, then be fixated back unto itself. The shoulder strap 14 has a hook tab 20 attached with a framed-X pattern stitch, overlapping the loop side of the shoulder strap 14 with the hook of the tab 20 facing the same direction of the shoulder strap 14 loop. This makes the strap adjustable by attaching back to itself across its entire length.

The back-strap 13 is constructed similar to the shoulder straps 14, only it has a hook tab 20 attached to both ends, instead of just one like the shoulder straps 14. The back strap 13 is applied by running one back-strap tab end 20 through the back-strap slide 16 on the rear of the left-arm cuff 11, and the other back-strap tab end 20 through the back-strap slide 16 on the rear of the right-arm cuff 12. The loop side of the strap should be facing outward, away from the patient's back, to allow back-strap 13 tab ends 20 to attach back to the back-strap. The left shoulder strap 14, the right shoulder strap 14 and the back-strap 13 each contain one loop side capable of receiving velcro hook, making the sling adjustable both horizontally, and vertically through four separate adjustment points.

FIG. D—The left-arm cuff 11 is constructed from an elastic woven band 21, which is elastic horizontally, but remains rigid vertically. Non-elastic cloth ends are attached to each end of the elastic woven band 21, in the form of one cuff-under edge 19 and one cuff-over edge 22. Cuff-under edge 19 is made by sewing two stacked pieces of cloth together on three edges, and then flipped inside out with open end rolled inward approximately an inch. Cuff-over edge 22 is made similarly to the cuff-under edge 19, only three cuff tongues 17 are sandwiched between the two pieces of cloth, with the hook sides of all three cuff tongues 17 facing the same direction, with approximately an inch of their elastic ends sticking out of the cloth. Once cuff-over edge 22 is sewn on three sides, it is flipped inside out with open end rolled inward about an inch. A U-shaped double-stitch secures the previously exposed elastic ends of the cuff tongues 17 which are now inside the cuff-over edge 22. Cuff-over edge 22 and cuff-under edge 19 are attached to elastic woven band 21 with similar U-shaped double-stitches, after ends of elastic woven band 21 have been inserted inside open ends of cuff-under edge 19 and cuff-over edge 22.

One quick-release buckle 15 is applied to elastic woven band 21 with non-elastic strapping which overlaps the elastic woven band 21 on both sides, placed next to cuff-under edge 19. One back-strap slide 16 is applied to elastic woven band 21 with non-elastic strapping which overlaps elastic woven band 21 on both sides, placed next to quick-release buckle at a 45-degree angle toward the center of the top of the elastic woven band 21. One shoulder strap 14 is attached to the exterior of the elastic woven band 21 by sewing the end without the hook tab 20 with a framed-X pattern stitch, with the shoulder strap's 14 loop side facing away from the elastic woven band 21.

1. a hemi-arm sling comprising of one semi-elastic hook receiving back strap to prevent forward shoulder roll and aid in posture restoration and two semi-elastic arm cuffs for comfortably holding on to each bicep so the weight of the injured arm is supported by the uninjured arm with the semi-elastic straps to reduce shoulder subluxation without putting pressure on the neck and the injured shoulder joint while allowing for full use and range of motion of both arms and hands, with each arm cuff having a semi-elastic hook receiving loop strap attached and also a quick release buckle to receive the hook receiving loop strap and a hard loop for receiving the back strap.
